(19) United States
(12) Patent Application Publication
(10) Pub. No.: US 2024/0371463 A1
(43) Pub. Date: Nov. 7, 2024

(54) METHODS FOR PREDICTING EPITOPE SPECIFICITY OF T CELL RECEPTORS

(71) Applicant: Universite de Lausanne, Lausanne (CH)

(72) Inventors: Vincent Zoete, Epalinges (CH); Marta Perez, Epalinges (CH); Francesca Mayol Rullan, Epalinges (CH)

(21) Appl. No.: 18/682,752

(22) PCT Filed: Aug. 30, 2022
(86) PCT No.: PCT/EP2022/074097

§ 371 Date: Feb. 9, 2024

Related U.S. Application Data
(60) Provisional application No. 63/239,253, filed on Aug. 31, 2021.

(51) Int. Cl. G16B 15/30; G16B 40/20

(52) U.S. Cl. CPC G16B 15/30; G16B 40/20

(57) ABSTRACT

This disclosure describes methods for predicting specificity of an immunological entity (e.g., T-cell receptor) to an epitope for cancer immunotherapy by clustering immunological entities using a metric derived from molecular fingerprints (e.g., physicochemical properties) and related to the molecular interactions that the most important residues of the immunological entity can perform. The resulting clusters correlate with the specificity of the immunological entities so that the members of the same cluster can potentially bind to the same or highly similar epitope(s). This disclosure provides opportunities for widely applicable high-precision adoptive T-cell therapy and personalized vaccination in oncology, while laying the foundation for deeper fundamental mechanistic understanding in tumor immunology in particular and immunology in general.

### CROSS-REFERENCE AND CLAIM OF PRIORITY

The present application is a national phase filing of International Application No. PCT/EP2022/074097 filed Aug. 30, 2022, which claims priority to U.S. Provisional Patent Application No. 63/239,253 filed Aug. 31, 2021. Both priority applications are incorporated herein by reference in their entirety.

### FIELD OF THE INVENTION

This invention relates to methods for predicting specificity of a T cell receptor (TCR) to an epitope.

### BACKGROUND OF THE INVENTION

Cancer immunotherapy has produced great advances recently. For further improvements, promising approaches like adoptive cell therapy or vaccination would require the precise knowledge of which T-cell receptor (TCR) recognizes which cancer epitope (i.e., peptide-MHC, pMHC) in the tumor of a given patient.

However, prediction of TCR specificities, the Holy Grail of immunoinformatics, is generally considered intractable due to the virtually unlimited number of pMHC and TCR sequences. Very few computational approaches have been successful at predicting TCR-pMHC interactions for uncharacterized epitopes. Several approaches to use protein structures and force field to model TCR-pMHC interactions have been attempted. Although this structure-based technology is powerful to optimize the affinity of a TCR towards a specific epitope, it has limited success for the prediction of interacting TCR:pMHC pairs at a large scale.

Accordingly, there remains a need for novel approaches that circumvent the limitations in the existing computational approaches.

### SUMMARY OF THE INVENTION

This disclosure addresses the need mentioned above in a number of aspects. In one aspect, this disclosure provides a method of identifying two immunological entities (e.g., TCR) as having similar epitope specificity. The method comprises: (a) selecting a subset of amino acids in a first immunological entity and a corresponding subset of amino acids in a second immunological entity, wherein the subset of amino acids in the first immunological entity and the corresponding subset of amino acids in the second immunological entity have an identical number of amino acids; (b) determining an amino acid sum of differences in each of a plurality of physicochemical properties by performing a pairwise comparison between an amino acid in the subset of amino acids in the first immunological entity and a corresponding amino acid in the corresponding subset of amino acids in the second immunological entity; (c) repeating steps (a) to (b) for remaining amino acids in the subset of amino acids in the first immunological entity and the corresponding subset of amino acids in the second immunological entity; (d) determining a subset sum of differences between the subset of amino acids in the first immunological entity and the corresponding subset of amino acids in the second immunological entity; (e) repeating steps (a) to (d) for one or more subsets of amino acids in the first immunological entity and the second immunological entity; (f) determining an aggregate value of all subset sums of differences between the first immunological entity and the second immunological entity by assigning a weight value to each of the subset sums; and (g) identifying the first immunological entity and the second immunological entity as having similar specificity to an epitope if the aggregate value is smaller than a threshold value.

In some embodiments, the immunological entity is a T cell receptor (TCR), a B cell receptor (BCR), an antibody, or a chimeric antigen receptor (CAR). In some embodiments, the immunological entity is a TCR. In some embodiments, the epitope is located on a peptide-MHC (pMHC).

In some embodiments, the subset of amino acids comprises 3 to 8 amino acids. In some embodiments, the subset of amino acids comprises 3 to 8 consecutive amino acids. In some embodiments, the subset of amino acids consists of 4 amino acids (e.g., 4 consecutive residues).

In some embodiments, one or more subsets of amino acids are selected from amino acids in CDR1α, CDR2α, CDR3α, CDR1β, CDR2β, and CDR₃α.

In some embodiments, step (f) determining an aggregate value in the method described herein comprises assigning a weight value of about 30% to the subset of amino acids in CDR3α or CDR3β. In some embodiments, step (f) determining an aggregate value comprises assigning a weight value of about 10% to the subset of amino acids in CDR1α, CDR2α, CDR1β, or CDR2β.

In some embodiments, the subset of amino acids does not include amino acids that are not solvent-exposed. In some embodiments, the subset of amino acids does not include amino acids in CDR1α, CDR2α, CDR1β, or CDR2β that have a relative solvent excluded surface area (SESA) of less than about 5%. In some embodiments, the subset of amino acids does not include amino acids in CDR3α or CDR3β that have a SESA of less than about 20%.

In some embodiments, the physicochemical properties comprise amino acid attributes selected from hydrophilicity value, polar requirement, long range nonbonded energy per atom, negative charge, positive charge, size, normalized relative frequency of bend, normalized frequency of β-turn, molecular weight, relative mutability, normalized frequency of coil, average volume of buried residue, conformational parameter of β-turn, residue volume, isoelectric point, optimized propensity to form reverse turn, chou-fasman parameter of coil conformation, information measure for loop, free energy in β-strand region, side chain volume, amino acid composition of total proteins, average relative probability of helix, α-helix indices, relative frequency of occurrence, helix-coil equilibrium constant, amino acid composition, number of codon(s), net charge, normalized frequency of turn, relative frequency in α-helix, average nonbonded energy per residue, bulkiness, normalized relative frequency of coil, refractivity, normalized frequency of left-handed α-helix, heat capacity, free energy in α-helical region, hydrophobicity factor, normalized frequency of extended structure, normalized frequency of β-sheet, unweighted, normalized frequency of β-sheet, information measure for pleated-sheet, hydropathy index, eisenberg hydrophobic index, average side chain orientation angle, average interactions per side chain atom, transfer free energy, and percentage of buried residues.

In some embodiments, the physicochemical properties comprise hydrophobicity, secondary structure propensity, size/mass, amino acid composition, codon degeneracy, and electrostatic charge.

In another aspect, this disclosure also provides a method of identifying a subset of immunological entities as having similar specificity to an epitope. The method comprises (i) providing a plurality of immunological entities; (ii) selecting two immunological entities from the set of immunological entities for pairwise comparison; (iii) identifying the two immunological entities as having similar specificity to an epitope according to the method as described herein; and (iv) repeating steps (ii) to (iii) for remaining immunological entities in the plurality of immunological entities and identifying a subset of immunological entities from the plurality of immunological entities as having similar specificity to the epitope.

In another aspect, this disclosure further provides a method of identifying two immunological entities as having similar specificity to an epitope. The method comprises: (i) providing a plurality of immunological entities; (ii) selecting two immunological entities from the set of immunological entities for pairwise comparison; (iii) performing steps (a) to (f) of the method as described herein; and (iv) repeating steps (ii) to (iii) for remaining immunological entities in the plurality of immunological entities and identifying a pair of immunological entities that have a minimum aggregate value among the plurality of immunological entities as the two immunological entities having similar specificity to the epitope.

In another aspect, this disclosure additionally provides a method of identifying a subset of immunological entities as having similar specificity to an epitope. The method comprises: (1) providing a plurality of immunological entities; (2) generating a similarity matrix for a subset of amino acids of an immunological entity of the set of immunological entities, wherein the similarity matrix comprises a plurality of physicochemical properties of each amino acid in the subset of amino acids; (3) repeating step (2) for one or more subsets of amino acids of the immunological entity; (4) repeating steps (2) to (3) for remaining immunological entities of the plurality of immunological entities; and (5) performing a clustering analysis based on a distance between two corresponding similarity matrices of a pair of immunological entities to identify a subset of immunological entities having similar specificity to the epitope.

In some embodiments, the distance is a Manhattan distance. In some embodiments, the clustering analysis comprises a hierarchical clustering. In some embodiments, the hierarchical clustering comprises an unweighted pair group method with arithmetic mean (UPGMA).

In another aspect, this disclosure also provides a method of identifying an epitope for a TCR that binds specifically to the epitope. The method comprises: identifying a second TCR that has similar specificity to an epitope with the TCR according to the method as described herein, wherein the second TCR binds specifically to a known epitope; and identifying the known epitope as an epitope to which the TCR binds specifically.

In yet another aspect, this disclosure also provides a method of identifying one or more TCRs that bind specifically to an epitope. The method comprises: selecting a candidate TCR that binds specifically to the epitope; identifying at least one TCR that has similar specificity to an epitope with the candidate TCR according to the method as described above; and identifying the at least one TCR as the one or more TCRs that bind specifically to the epitope.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 shows a representative scheme for describing amino acids using the 5 Atchley factors as descriptors rather than by sequence.

FIG. 2 shows encoding a protein loop (e.g., a TCR CDR3β loop) into a series of 4×5 matrices, each matrix corresponding to a set of 4 consecutive amino acids described by 5 Atchley factors.

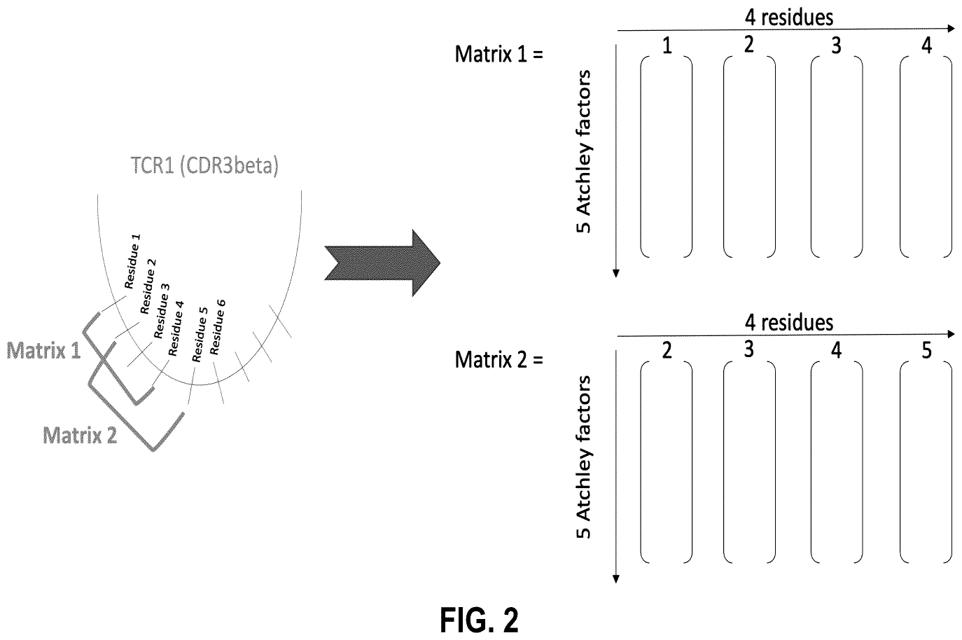

FIG. 3 shows hierarchical clustering of a set of 54 TCRs recognizing 16 different pMHC using the Achtley-based distance considering only sliding windows of 4 consecutive residues of the CDR3β. After clustering, each TCR is colored according to the pMHC it binds. The sequence of the bound peptide is also given.

FIG. 4 shows hierarchical clustering of a set of 54 TCRs recognizing 16 different pMHC using the Achtley-based distance considering all 6 TCR CDRs (i.e., CDR1α, CDR2α, CDR3α, CDR1β, CDR2β, and CDR3β). After clustering, each TCR is colored according to the pMHC it binds. The sequence of the bound peptide is also given.

FIG. 5 shows that buried residues are excluded from the distance calculation. Due to the buriedness of some of the residues in the loop, the CASN, ASNP, SNPG, NPGL, HNEQ, NEQF, and EQFF 4-residues sliding windows were excluded from the calculation distance. However, the HNEF quadruplet of consecutive solvent-exposed residues was added to the analysis.

FIG. 6 shows hierarchical clustering of a set of 54 TCRs recognizing 16 different pMHC using the Achtley-based distance considering all 6 TCR CDRs (i.e., CDR1α, CDR2α, CDR3α, CDR1β, CDR2β, and CDR3β), as well as residue buriedness. After clustering, each TCR is colored according to the pMHC it binds. The sequence of the bound peptide is also given.

FIG. 7 shows hierarchical clustering of a set of 374 TCRs recognizing 39 different pMHC using the Achtley-based distance considering, only the CDR3β residues (left), or all 6 TCR CDRs (i.e., CDR1α, CDR2α, CDR3α, CDR1β, CDR2β, and CDR3β) (middle), as well as residue buriedness (right). After clustering, each TCR is colored according to the pMHC it binds. The sequence of the bound peptide is also given.

FIG. 8 shows an application of the disclosed Achtley-based TCR-distance calculation and clustering to the prediction of specificity of orphan TCRs. Orphan TCRs (without known pMHC specificity) are clustered together with a large number of TCR for which the cognate pMHC is known. Given the fact that the clustering approach tends to group together TCRs that bind the same pMHC, orphan TCRs could be tested experimentally for their ability to bind pMHC known to interact with TCRs close to the orphan ones in the hierarchical clustering.

### DETAILED DESCRIPTION OF THE INVENTION

This disclosure describes methods for predicting epitope specificity of an immunological entity (e.g., T-cell receptor) for cancer immunotherapy by clustering immunological entities using a metric derived from molecular fingerprints (e.g., physicochemical properties) and related to the molecular interactions that the most important residues of the immunological entity can perform. The resulting clusters correlate with the specificity of the immunological entities so that the members of the same cluster can potentially bind to the same or highly similar epitope(s). This disclosure provides opportunities for widely applicable high-precision adoptive T-cell therapy and personalized vaccination in oncology, while laying the foundation for deeper fundamental mechanistic understanding in tumor immunology in particular and immunology in general.

In particular, this disclosure demonstrates that predicting actual TCR-pMHC interactions can be achieved by encoding the 3D structures of pMHC and TCR, e.g., the complex shape, charge, and lipophilicity spatial distributions of molecules, into simple one-dimensional vectors (i.e., fingerprints). As a result, comparing molecular shapes reduces to calculating distances between vectors, which can be achieved in seconds for millions of possible complexes. In addition, the methods as disclosed are less sensitive to uncertainties in atomic spatial coordinates and could remain efficient when applied to homology models. Moreover, being essentially physics-based, the disclosed methods, unlike existing sequence-based approaches, will require a limited amount of experimental data for the training.

In one aspect, this disclosure provides a method of identifying two immunological entities as having similar specificity to an epitope. The method comprises: (a) selecting a subset of amino acids in a first immunological entity and a corresponding subset of amino acids in a second immunological entity, wherein the subset of amino acids in the first immunological entity and the corresponding subset of amino acids in the second immunological entity have an identical number of amino acids; (b) determining an amino acid sum of differences in each of a plurality of physicochemical properties by performing a pairwise comparison between an amino acid in the subset of amino acids in the first immunological entity and a corresponding amino acid in the corresponding subset of amino acids in the second immunological entity; (c) repeating steps (a) to (b) for remaining amino acids in the subset of amino acids in the first immunological entity and the corresponding subset of amino acids in the second immunological entity; (d) determining a subset sum of differences between the subset of amino acids in the first immunological entity and the corresponding subset of amino acids in the second immunological entity; (e) repeating steps (a) to (d) for one or more subsets of amino acids in the first immunological entity and the second immunological entity; (f) determining an aggregate value of all subset sums of differences between the first immunological entity and the second immunological entity by assigning a weight value to each of the subset sums; and (g) identifying the first immunological entity and the second immunological entity as having similar specificity to an epitope if the aggregate value is smaller than a threshold value.

As used herein, “immunological entity” refers to any substance responsible for an immune reaction. Immunological entities include antibodies, antigen binding fragments of an antibody, T cell receptors, fragments of a T cell receptor, B cell receptors, fragments of a B cell receptor, chimeric antigen receptors (CAR), cells comprising one or more of them (e.g., T cells comprising a chimeric antigen receptor (CAR) (CAR-T)), and the like.

In some embodiments, the immunological entity is a T cell receptor (TCR), a B cell receptor (BCR), an antibody, or a chimeric antigen receptor (CAR). In some embodiments, the immunological entity is a TCR. In some embodiments, the epitope is located on peptide-MHC (pMHC).

As used herein, “antibody” is used in the same meaning that is commonly used in the art and refers to a protein reacting highly specifically to an antigen, which is made in the immune system when the antigen contacts the biological immune system (antigen stimulation). Antibodies may be of any origin, type, shape, or the like, as long as the antibody binds to the specific epitope. The antibodies described herein can be divided into framework regions and antigen binding regions (CDR).

As used herein, “T cell receptor (TCR)” is also called a T cell antigen receptor. A T cell receptor refers to a receptor recognizing an antigen, expressed on a cell membrane of a T cell that plays a central role in the immune system. TCRs have an α chain, β chain, γ chain, and δ chain, with which an αβ or γδ dimer is constituted. TCRs consisting of the combination of the former are called αβTCRs, and TCRs consisting of the combination of the latter are called γδ TCRs. T cells having such TCRs are respectively called αβ T cells and γϵT cells. The TCRs are structurally very similar to a Fab fragment of an antibody produced by B cells and recognize antigen molecules bound to an MHC molecule. Since a TCR gene of a mature T cell has undergone gene rearrangement, an individual has highly diverse TCRs that enable recognition of various antigens. TCRs also form a complex by binding to a non-variable CD3 molecule at the cell membrane. CD3 has an amino acid sequence called ITAM (immunoreceptor tyrosine-based activation motif) in the intracellular region. This motif is considered to be involved in intracellular signaling. Each TCR chain is comprised of a variable domain (V) and a constant domain (C). A constant domain has a short cytoplasm section penetrating the cell membrane. A variable domain is present outside the cell and binds to an antigen-MHC complex. A variable domain has three hypervariable domains or regions called complementarity-determining regions (CDRs), which bind to an antigen-MHC complex. The three CDRs are called CDR1, CDR2, and CDR3. TCR gene rearrangement is similar to the process of β cell receptors known as immunoglobulins. For gene rearrangement of aß TCRs, VDJ recombination of β chain is performed, followed by VJ recombination of an α chain. When the α chain is rearranged, the gene of the δ chain is deleted from the chromosome. Thus, a T cell having an αβ TCR would never have a γδ TCR simultaneously. In contrast, a signal via a γδ TCR in a T cell having the TCR suppresses the expression of β chain, so that a T cell having a γδ TCR would never have an αβ TCR simultaneously.

As used herein, “B cell receptor (BCR)” is also called a B cell antigen receptor, referring to those comprised of Igα/Igβ (CD79a/CD79b) heterodimer (α/β) associated with a membrane-bound immunoglobulin (mIg) molecule. A mIg subunit binds to an antigen to induce aggregation of receptors, while an α/β subunit transmits a signal toward the cell. Aggregation of BCRs is understood to quickly activate Lyn, Blk, and Fyn of a Src family kinase in the same manner as Syk and Btk of tyrosine kinase. Many different results are produced depending on the complexity of BCR signaling. Examples thereof include survival, resistance (allergy, lack of hypersensitive reaction to an antigen) or apoptosis, cell division, differentiation into an antibody producing cell or memory B cell, and the like. Many hundreds of million types of T cells with different sequences of the variable regions of TCRs are produced, and many hundreds of million types of B cells with different sequences of the variable regions of BCRs (or antibodies) are produced. Since the individual sequences of TCRs and BCRs vary due to rearrangement or mutation of the genomic sequence, a clue for antigen specificity of a T cell or B cell can be found by determining the sequence of mRNA (cDNA) or the genomic sequence of TCR/BCR.

As used herein, “chimeric antigen receptor (CAR)” is a collective term for chimeric proteins having a single chain antibody (scFv) having a light chain (VL) and a heavy chain (VH) of a tumor antigen specific monoclonal antibody variable region bound in series on the N-terminus side, and a T cell receptor (TCR) ζ chain on the C-terminus side. A chimeric antigen receptor is an artificial T cell receptor used in gene and cell therapy, in which an artificial T cell receptor that is genetically engineered to defeat the immune evasion mechanism of tumor is transfected into patient T cells, which are amplified and cultured outside the body and then injected into a patient (Dotti G, et al., Hum Gene Ther 20:1229-1239, 2009). Such a CAR can be produced using an epitope that is identified or clustered by the present invention. Gene and cell therapy can be materialized using the produced CAR or genetically modified T cells comprising such a CAR (see Brentjens R, et al. Nat Rev Clin Oncol. 2016 13, 370-383).

As used herein, “epitope” refers to a site in a molecule of an immunological entity binder (e.g., antigen), to which an immunological entity such as an antibody or a lymphocyte receptor (TCR, BCR, or the like) binds. While a straight chain of an amino acid can constitute an epitope (strain chain epitope), separated sites of a protein can constitute a stereo structure to function as an epitope (conformational epitope). Epitopes of the invention are not limited by such detailed classification of epitopes. It is understood that if certain immunological entities such as antibodies have the same epitope, an immunological entity such as a TCR or an antibody having another sequence can also be used in the same manner.

As used herein, “similarity” refers to the degree molecules are similar for molecules such as an immunological entity binder (e.g., antigen) or epitope or a part thereof. Similarity can be determined based on a difference in physicochemical properties. Generally, the concept encompasses a broadly defined “structural similarity”. Although not wishing to be bound by any theory, it is understood that antibodies, TCRs, BCRs, or the like binding to an epitope belonging to an identical cluster can be assigned to a disease, disorder, symptom, physiological phenomenon, or the like in the same category when immunological entities or epitopes are classified based on such similarity in some of the embodiments of the present invention. Therefore, a variety of diagnosis (incidence of cancer, compatibility of administered drug, and the like) is made possible by studying whether there are antibodies, TCRs, BCRs, or the like that react to the same epitope cluster by using the disclosed methods.

As used herein, a “corresponding” amino acid refers to an amino acid which has, or is expected to have, in a certain polypeptide molecule, similar action as a predetermined amino acid in a benchmark polypeptide, and for enzyme molecules, refers to an amino acid which is present at a similar position in an active site and makes a similar contribution to catalytic activity. It is preferable to define identical residues when investigating a corresponding amino acid. A corresponding amino acid can be a specific amino acid subjected to, for example, cysteination, glutathionylation, S-S bond formation, oxidation (e.g., oxidation of methionine side chain), formylation, acetylation, phosphorylation, glycosylation, myristylation, or the like. Alternatively, a corresponding amino acid can be an amino acid responsible for dimerization. Such a “corresponding” amino acid or nucleic acid may be a region or a domain (e.g., V region, D region, or the like) over a certain range. Thus, it is referred to herein as a “corresponding” region, subset, or domain in such a case.

In some embodiments, the subset of amino acids comprises 3 to 10 (e.g., 3, 4, 5, 6, 7, 8, 9, 10) amino acids. In some embodiments, the subset of amino acids comprises 3 to 10 (e.g., 3, 4, 5, 6, 7, 8, 9, 10) consecutive amino acids. In some embodiments, the subset of amino acids consists of 4 amino acids (e.g., 4 consecutive residues).

In some embodiments, it is important for predicting epitope specificity by taking into account the contribution of residues in all CDRs (e.g., CDR1α, CDR2α, CDR3α, CDR1β, CDR2β, and CDR3β of a TCR). Accordingly, In some embodiments, one or more subsets of amino acids are selected from amino acids in CDR1α, CDR2α, CDR3α, CDR1β, CDR2β, and CDR3β.

In some embodiments, step (f) determining an aggregate value in the method described herein comprises assigning a weight value of about 10% to about 35% (e.g., about 20%, about 25%, about 30%, about 35%) to the subset of amino acids in CDR3α or CDR3β. In some embodiments, step (f) determining an aggregate value comprises assigning a weight value of about 5% to about 15% (e.g., about 5%, about 8%, about 10%, about 12%, about 14%, about 15%) to the subset of amino acids in CDR1α, CDR2α, CDR1β, or CDR2β.

In some embodiments, the subset of amino acids does not include amino acids that are not solvent-exposed. In some embodiments, the subset of amino acids does not include amino acids in CDR1α, CDR2α, CDR1β, or CDR2β that have a relative solvent excluded surface area (SESA) of less than about 3% to about 8% (e.g., about 3%, about 4% about, 5%, about 6%, about 7%, about 8%). In some embodiments, the subset of amino acids does not include amino acids in CDR3α or CDR3β that have a SESA of less than about 15% to about 25% (e.g., about 15%, about 18%, about 20%, about 22%, about 25%).

In some embodiments, the physicochemical properties comprise amino acid attributes selected from hydrophilicity value, polar requirement, long range nonbonded energy per atom, negative charge, positive charge, size, normalized relative frequency of bend, normalized frequency of β-turn, molecular weight, relative mutability, normalized frequency of coil, average volume of buried residue, conformational parameter of β-turn, residue volume, isoelectric point, optimized propensity to form reverse turn, chou-fasman parameter of coil conformation, information measure for loop, free energy in β-strand region, side chain volume, amino acid composition of total proteins, average relative probability of helix, α-helix indices, relative frequency of occurrence, helix-coil equilibrium constant, amino acid composition, number of codon(s), net charge, normalized frequency of turn, relative frequency in α-helix, average nonbonded energy per residue, bulkiness, normalized relative frequency of coil, refractivity, normalized frequency of left-handed α-helix, heat capacity, free energy in α-helical region, hydrophobicity factor, normalized frequency of extended structure, normalized frequency of β-sheet, unweighted, normalized frequency of β-sheet, information measure for pleated-sheet, hydropathy index, eisenberg hydrophobic index, average side chain orientation angle, average interactions per side chain atom, transfer free energy, and percentage of buried residues.

In some embodiments, the physicochemical properties comprise hydrophobicity, secondary structure propensity, size/mass, amino acid composition, codon degeneracy, and electrostatic charge.

In another aspect, this disclosure also provides a method of identifying a subset of immunological entities as having similar specificity to an epitope. The method comprises (i) providing a plurality of immunological entities; (ii) selecting two immunological entities from the set of immunological entities for pairwise comparison; (iii) identifying the two immunological entities as having similar specificity to an epitope according to the method as described herein; and (iv) repeating steps (ii) to (iii) for remaining immunological entities in the plurality of immunological entities and identifying a subset of immunological entities from the plurality of immunological entities as having similar specificity to the epitope.

In another aspect, this disclosure further provides a method of identifying two immunological entities as having similar specificity to an epitope. The method comprises: (i) providing a plurality of immunological entities; (ii) selecting two immunological entities from the set of immunological entities for pairwise comparison; (iii) performing steps (a) to (f) of the method as described herein; and (iv) repeating steps (ii) to (iii) for remaining immunological entities in the plurality of immunological entities and identifying a pair of immunological entities that have a minimum aggregate value among the plurality of immunological entities as the two immunological entities having similar specificity to the epitope.

In another aspect, this disclosure additionally provides a method of identifying a subset of immunological entities as having similar specificity to an epitope. The method comprises: (1) providing a plurality of immunological entities; (2) generating a similarity matrix for a subset of amino acids of an immunological entity of the set of immunological entities, wherein the similarity matrix comprises a plurality of physicochemical properties of each amino acid in the subset of amino acids; (3) repeating step (2) for one or more subsets of amino acids of the immunological entity; (4) repeating steps (2) to (3) for remaining immunological entities of the plurality of immunological entities; and (5) performing a clustering analysis based on a distance between two corresponding similarity matrices of a pair of immunological entities to identify a subset of immunological entities having similar specificity to the epitope.

As used herein, the term “clustering analysis” refers to using a clustering algorithm to identify groups of members that are similar. It divides a data set so that records with similar content are in the same group, and groups are as different as possible from each other. When the categories are unspecified, this is sometimes referred to as unsupervised clustering. When the categories are specified a priori, this is sometimes referred to as supervised clustering.

In some embodiments, the clustering algorithms comprise partitional clustering, hierarchical clustering, k-nearest neighbor (KNN), K-means and fuzzy clustering, and Kohonen self-organizing maps clustering. In some embodiments, clustering includes agglomerative “bottom-up” or divisive “top-down” hierarchical clustering, distance “partition” clustering, and alignment clustering.

In some embodiments, the clustering analysis comprises a hierarchical clustering. In some embodiments, the hierarchical clustering comprises an unweighted pair group method with arithmetic mean (UPGMA).

In some embodiments, the distance is a Manhattan distance. In some embodiments, the distance is a normalized distance obtained, e.g., by dividing each of them by the maximum calculated value for all possible pairs.

In some embodiments, the distance between two TCRs ranges from 0 (e.g., for two totally unrelated TCRs) to 1 (e.g., for two TCRs bearing an identical set of 4 residues on each considered loop, e.g., only CDR3β or all CDRs). In some embodiments, a threshold value can be used as an indicator to classify the similarity between TCRs. For example, a pair with a maximum distance found by clustering analysis using a hierarchical clustering methodology (e.g., group average method (average linkage clustering), nearest neighbor method (NN method), K-NN method, Ward method, furthest neighbor method, or centroid method) of less than a specific value can be deemed to be in identical cluster. Examples of such a value include, but are not limited to, less than 1, less than 0.95, less than 0.9, less than 0.85, less than 0.8, less than 0.75, less than 0.7, less than 0.65, less than 0.6, less than 0.55, less than 0.5, less than 0.45, less than 0.4, less than 0.35, less than 0.3,less than 0.25, less than 0.2, less than 0.15, less than 0.1, less than 0.05, and the like. In some embodiments, a specific threshold value can be set for evaluation. For example, about 0.9 can be used to distinguish whether an entity belongs to an identical group or another group. To increase the degree of separation, the threshold value can be appropriately raised. When, for example, about 0.9 is used, the threshold value can be set higher to about 0.95 or the like.

In another aspect, this disclosure also provides a method of identifying an epitope for a TCR that binds specifically to the epitope. The method comprises: identifying a second TCR that has similar specificity to an epitope with the TCR according to the method as described herein, wherein the second TCR binds specifically to a known epitope; and identifying the known epitope as an epitope to which the TCR binds specifically.

In yet another aspect, this disclosure also provides a method of identifying one or more TCRs that bind specifically to an epitope. The method comprises: selecting a candidate TCR that binds specifically to the epitope; identifying at least one TCR that has similar specificity to an epitope with the candidate TCR according to the method as described above; and identifying at least one TCR as the one or more TCRs that bind specifically to the epitope.

### Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “k-nearest neighbor (KNN)” as used herein refers to a classification method that classifies a point by calculating the distances between the point and points in the training data set. Then it assigns the point to the class that is most common among its k-nearest neighbors (where k is an integer).

The term “machine learning,” as used herein, refers to a computer algorithm used to extract useful information from a database by building probabilistic models in an automated way.

The term “regression tree,” as used herein, refers to a decision tree that predicts values of continuous variables.

The term “supervised learning,” as used herein, refers to a data analysis using a well-defined (known) dependent variable. All regression and classification algorithms are supervised. In contrast, “unsupervised learning” refers to the collection of algorithms where groupings of the data are defined without the use of a dependent variable.

The term “test data” refers to a data set independent of the training data set, used to evaluate the estimates of the model parameters (i.e., weights).

As used herein, the term “clustering tree” refers to a hierarchical tree structure in which observations, such as organisms, genes, and polynucleotides, are separated into one or more clusters. The root node of a clustering tree consists of a single cluster containing all observations, and the leaf nodes correspond to individual observations. A clustering tree can be constructed on the basis of a variety of characteristics of the observations. Many techniques known in the art, e.g., hierarchical clustering analysis, can be used to construct a clustering tree. A non-limiting example of the clustering tree is a phylogenetic, taxonomic or evolutionary tree.

As used herein, “gene region” refers to a framework region, antigen binding region (CDR), and each of the regions such as the V region, D region, J region, and C region. Such gene regions are known in the art and can be appropriately determined by referring to a database or the like. As used herein, “homology” of genes refers to the degree of identity of two or more gene sequences to one another. Generally, having “homology” refers to having a high degree of identity or similarity. Therefore, two genes having higher homology have higher identity or similarity of the sequences thereof. Whether two genes have homology can be found by direct comparison of sequences, or by hybridization under stringent conditions for nucleic acids. As used herein, “homology search” refers to a search for homology. For example, homology can be searched in silico using a computer. As used herein, “V region” refers to a variable domain (V) region of a variable region of an immunological entity such as an antibody, TCR, or BCR. As used herein, “D region” refers to a D region of a variable region of an immunological entity such as an antibody, TCR, or BCR. As used herein, “J region” refers to a J region of a variable region of an immunological entity such as an antibody, TCR, or BCR. As used herein, “C region” refers to a constant domain (C) region of an immunological entity such as an antibody, TCR, or BCR. As used herein, “repertoire of a variable region” refers to a collection of V(D)J regions optionally created by gene rearrangement in TCR or BCR. The phrases TCR repertoire, BCR repertoire, and the like are used, but they can also be called, for example, T cell repertoire, B cell repertoire, or the like. For example, “T cell repertoire” refers to a collection of lymphocytes characterized by the expression of a T cell receptor (TCR) serving an important role in antigen recognition or recognition of an immunological entity binder. Since a change in T cell repertoire is a significant indicator of an immune state in a diseased state or physiological state, T cell repertoire analysis has been performed for identification of antigen specific T cells involved in the development of a disease and diagnosis of T lymphocyte abnormalities.

As used herein, whether epitopes are “identical” or “different” can be determined by similarity in physicochemical properties in accordance with the clustering as disclosed. “Identical” does not refer to complete identity of amino acid sequences, but refers to substantially the same quality of physicochemical properties. Epitopes belonging to an identical cluster are determined as “identical” in this disclosure and considered as having similar epitope specificity. Therefore, “different” epitopes refer to epitopes that do not belong to the “identical” cluster. In one embodiment, it can be determined whether they belong to an identical cluster depending on whether the epitopes are “identical” or “different.” When performing cluster analysis, an immunological entity (e.g., TCR) is, in comparison to another immunological entity, determined to be identical if belonging to the same cluster and determined to be different if belonging to a different cluster. Similar, epitopes to which immunological entities in an identical cluster bind can be classified into an identical cluster to generate the cluster. Immunological entities can also be evaluated for at least one endpoint selected from the group consisting of properties and similarity with a known immunological entity thereof to perform the cluster classification by targeting an immunological entity meeting a predetermined baseline.

As used herein, “protein,” “polypeptide,” “oligopeptide,” and “peptide” are used herein to have the same meaning and refer to a polymer of amino acids with any length. The polymer may be straight, branched, or cyclic. An amino acid may be a naturally occurring, non-naturally occurring, or modified amino acid. The term may also encompass those assembled into a complex of multiple polypeptide chains. The term also encompasses naturally occurring or artificially modified amino acid polymers. Examples of such a modification include disulfide bond formation, glycosylation, lipidation, acetylation, phosphorylation, and any other manipulation or modification (e.g., conjugation with a labeling component). The definition also encompasses, for example, polypeptides comprising one or more analogs of an amino acid (e.g., including non-naturally occurring amino acids and the like), peptide-like compounds (e.g., peptoids), and other known modifications in the art.

As used herein, “polynucleotide,” “oligonucleotide,” and “nucleic acid” are used herein to have the same meaning and refer to a polymer of nucleotides with any length. The term also encompasses “oligonucleotide derivative” and “polynucleotide derivative”. “Oligonucleotide derivative” and “polynucleotide derivative” refer to an oligonucleotide or polynucleotide that comprises a nucleotide derivative or has a bond between nucleotides which is different from normal. The terms are used interchangeably. Specific examples of such an oligonucleotide include 2′-O-methyl-ribonucleotide, oligonucleotide derivatives having a phosphodiester bond in an oligonucleotide converted to a phosphorothioate bond, oligonucleotide derivatives having a phosphodiester bond in an oligonucleotide converted to an N3′-P5′ phosphoramidate bond, oligonucleotide derivatives having ribose and phosphodiester bond in an oligonucleotide converted to a peptide nucleic acid bond, oligonucleotide derivatives having uracil in an oligonucleotide replaced with C-5 propinyluracil, oligonucleotide derivatives having uracil in an oligonucleotide replaced with C-5 thiazoluracil, oligonucleotide derivatives having cytosine in an oligonucleotide replaced with C-5 propinylcytosine, oligonucleotide derivatives having cytosine in an oligonucleotide replaced with phenoxazine-modified cytosine, oligonucleotide derivatives having ribose in DNA replaced with 2′-O-propylribose, oligonucleotide derivatives having ribose in an oligonucleotide replaced with 2′-methoxyethoxyribose, and the like. Unless noted otherwise, specific nucleic acid sequences are also intended to encompass conservatively modified variants (e.g., degenerate codon substitute) and complement sequences in the same manner as the expressly shown sequences. Specifically, degenerate codon substitutes can be achieved by preparing a sequence with the third position of one or more selected (or all) codons substituted with a mixed base and/or deoxyinosine residue (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). As used herein, “nucleic acid” is used interchangeably with a gene, cDNA, RNA, an oligonucleotide, and polynucleotide. As used herein, a “nucleotide” may be naturally occurring or non-naturally occurring.

As used herein, “diagnosis” refers to identification of a variety of parameters associated with a disease, disorder, state, or the like in a subject to judge the current or future status of such a disease, a disorder, a state, or the like. By using the method, the apparatus, or the system of the present invention, the state in the body can be examined. A variety of parameters such as a disease, a disorder, or a state in a subject, a formulation or a method for treatment or prevention to be administered can be selected using such information. As used herein, in a narrow sense, “diagnosis” refers to diagnosis of the current status, while encompassing “early diagnosis”, “presumptive diagnosis,” “advance diagnosis,” and the like in a broad sense. Since the diagnosis method of the invention, in principle, can utilize what has come from a body and can be implemented without a healthcare professional such as a doctor, the method is industrially useful. As used herein, “presumptive diagnosis, advance diagnosis, or diagnosis” in particular may be called “assistance” in order to clarify that the method can be implemented without a healthcare professional such as a doctor.

Thus, as used herein, an “agent” (or a detection agent or the like) which “specifically” interacts with (or binds to) a biological agent such as a polynucleotide or a polypeptide includes an agent whose affinity to the biological agent such as a polynucleotide or a polypeptide is typically equal to or higher than, preferably significantly (e.g., statistically significantly) higher than the affinity to other unrelated polynucleotide or polypeptide (particularly those with less than 30% identity). Such affinity can be measured, for example, by a hybridization assay, a binding assay, or the like.

As used herein, a first substance or agent “specifically” interacting with (or binding to) a second substance or agent refers to a first substance or agent interacting with (or binding to) the second substance or agent with higher affinity than that to a substance or agent other than the second substance or agent (particularly another substance or agent that is present in a sample containing the second substance or agent). Examples of interaction (or bond) specific to a substance or an agent include, but are not limited to, a ligand-receptor reaction, hybridization in nucleic acids, an antigen-antibody reaction in proteins, an enzyme-substrate reaction, and when both a nucleic acid and a protein are involved, a reaction between a transcription factor and a binding site of the transcription factor and the like, protein-lipid interaction, nucleic acid-lipid interaction, and the like. Thus, when both of the substances or agents are nucleic acids, a first substance or agent “specifically interacting” with a second substance or agent encompasses the first substance or agent having complementarity to at least a part of the second substance or agent. For example, when both of the substances or agents are proteins, examples of “specific” interaction (or bond) of a first substance or agent with a second substance or agent include, but are not limited to, interaction by an antigen-antibody reaction, interaction by a receptor-ligand reaction, enzyme-substrate interaction, and the like. When two kinds of substances or agents include a protein and a nucleic acid, “specific” interaction (or bond) of a first substance or agent with a second substance or agent encompasses interaction (or bond) between a transcription factor and a binding region of a nucleic acid molecule which is a target of the transcription factor.

The term “immune response,” as used herein, refers to any type of immune response, including, but not limited to, innate immune responses (e.g., activation of Toll receptor signaling cascade), cell-mediated immune responses (e.g., responses mediated by T cells (e.g., antigen-specific T cells) and non-specific cells of the immune system) and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

The term “disease” as used herein is intended to be generally synonymous and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, descriptions of “first,” “second,” etc. (“third” . . . and the like) indicate that entities are different from each other.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of this disclosure.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges are meant to be encompassed within the scope of the present disclosure. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of this disclosure unless otherwise claimed. No language in the specification should be construed as indicating any non-claimed element as essential to the practice of this disclosure.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

### TCR PDB Structures

The methods were developed using TCR PDB structures and VDJdb TCR sequences and validated using TCR sequences provided by A. Harari/Coukos lab. In each set, TCR duplicates (same CDR 1, 2, 3 α and β composition) and singleton peptides (peptides existing one single time and therefore not having a pair to cluster) were removed. 54 TCRs in the PDB set, 374 TCR in the VDJdb set, and 58 TCRs were used in the validation set.

### TCR Structure Modeling of the VDJdb TCR Sequences and TCR Sequences

The Rosetta “TCRmodel” protocol was adapted to the disclosed methods and applied to find the respective templates and model TCR. A total of 10 models were produced for each TCR, and the lowest energy one using Rosetta energy function as implemented in Rosetta was taken as TCR structural model.

### Solvent Accessibility calculation of each CDR1α, CDR2α, CDR3α, CDR1β, CDR2β and CDR3β Residue in Each TCR Structure

The solvent accessibility of each CDR1α, CDR2α, CDR3α and CDR1β, CDR2β and CDR3β residue was determined as the relative solvent excluded surface area (SESA) computed with the MSMS package of the UCSF Chimera software. SESA was calculated by normalizing the surface area of the residue in the TCR of interest by its surface area in a reference state.

### Hierarchical Clustering of TCRs

A computational pipeline was implemented based on a biophysical approach that allows TCR comparisons by analyzing the physicochemical properties (e.g., biophysicochemical properties) of the 4-mer subunits that are possible to construct from CDR1α, CDR2α, CDR3α, CDR1β, CDR2β, and CDR3β and comparing them across all the TCRs. To provide insights into the clusters, structural models were created for the TCRs as described in the previous section. The clustering pipeline consists of four main steps. First, all possible sliding windows of 4 residues that constitute the so-called 4-mer subunits were identified. The CDR residues that may not contact the peptide as determined by the solvent accessibility in the structural models were excluded from this process because these residues usually do not contact the HLA peptide. In some examples, only the residues of CDR1s and CDR2s (a and B) with SESA>5% are considered, and for CDR3s (α and β) just the residues with SESA>20% are considered.

Next, each 4-mer subunit was converted into a biophysicochemical representation using 5 Atchley factors that describe i) hydrophobicity, ii) secondary structure, iii) size/mass, iv) codon degeneracy, and v) electric charge. For each CDR of a pair of TCRs, all the n 4-mer subunits that are possible to construct from the first TCR with all the m possible 4-mer subunits of the second TCR were compared. This results in n*m matrices to compare for each CDR for each pair of TCRs. The matrices' comparison was performed via a Manhattan distance score normalized over the maximum possible distance. This score ranges from 0, for 4-mers sharing exactly the same biophysicochemical properties, to 1, for 4-mers that have totally different biophysiochemical properties.

A distance tree was constructed using the smallest distance for each TCR pair. In some examples, for the TCR pair distance calculation, the weighting contribution for CDR1s and CDR2s (α and β) was 10%, and the weighting for CDR3s (α and β) was 30%. It is of note that the contribution of all the CDRs was taken into account by giving higher weights to CDRs that dominate the contact with the peptide. The generic hierarchical clustering algorithm UPGMA (unweighted pair group method with arithmetic mean) was used.

### Example 2

Encoding residues and loops using Atchley factors

The disclosed approach is based, in part, on clustering immunological entities, e.g., TCRs, using a metric related to the molecular interactions that the most important residues of this receptor can perform. The resulting clusters correlate with the specificity of the TCRs, such that the TCRs members of the same cluster can potentially bind to the same pMHC complex or to very similar ones.

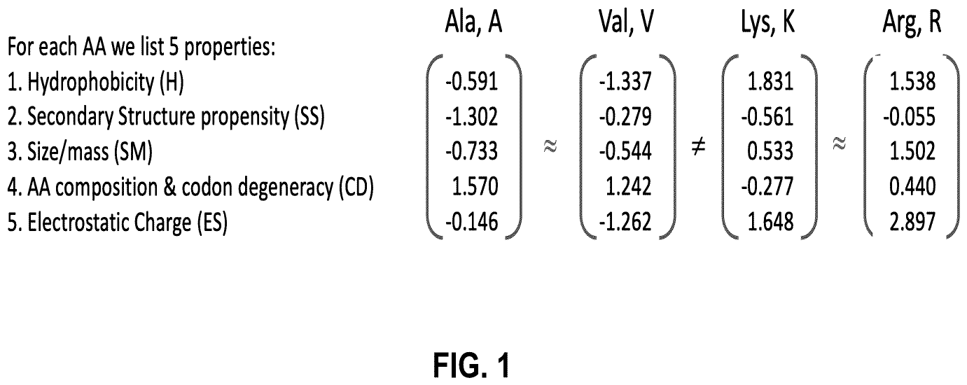

An important aspect of the disclosed approach is to describe each important TCR residue, not by its nature (like Ala, Val, Lys or Arg), but using its physicochemical descriptors encoded in the main Atchley factors (e.g., 5 main Atchley factors) (Atchley, W. R., et al. PNAS 102, 6395-6400 (2005)) (FIG. 1). For example, a set of 4 consecutive residues is encoded as a matrix of 4columns and 5 lines, each column corresponding to a residue, and each line to the value of a given Atchley factor for the 4 residues.

Given that, in TCRs, the most important residues for the interaction with a pMHC are present in the CDR1α, CDR2α, CDR3α, CDR1β, CDR2β, and CDR3β and can contain more than four residues, a given loop can be represented by a series of several 4×5 matrices (each one corresponding to a sliding window of 4 consecutive residues) to explore the most important part of the CDR sequence.

### Calculating Distances Between CDRs and TCRs

Given the encoding of residues using Atchley factors, it is possible to calculate the distance between two sets of 4 consecutive residues as the Manhattan distance between the two corresponding matrices M1 and M2, i.e., d (M1, M2) where:

$$d ( M 1 , M 2 ) = {\sum }_{i = [ 1 : 4 ]} \vert {H}_{1 , i} - {H}_{2 , i} \vert + \vert {SS}_{1 , i} - S {S}_{2 , i} \vert + \vert {SM}_{1 , i} - S {M}_{2 , i} \vert + \vert {CD}_{1 , i} - C {D}_{2 , i} \vert + \vert {ES}_{1 , i} - E {S}_{2 , i} \vert$$

Here, H_{1,i} is the Hydrophobicity Atchley factor of residue i in Matrix 1, H_{2.i} is the Hydrophobicity Atchley factor of residue i in Matrix 1, SS₁ᵢ is the Secondary Structure Propensity Atchley factor of residue i in Matrix 1, etc.

To calculate the distance between two corresponding loops, such as the CDR3β of two different TCRs, the n possible 4-residues sliding windows of the first TCR and the m 4-residues sliding windows of the second TCR were used. Then, the distance between each possible corresponding pairs of matrices was calculated. The smallest distance, dₘᵢₙ, between all possible pairs of matrices was retained as the distance between the two CDR3β loops.

$$d ( TCR 1 CDR 3 b , TCR 2 CDR 3 b ) = min ( {d ( {M}_{i} , {M}_{j} )}_{i ∈ [ 1 , n ] , j ∈ [ 1 , m ]} )$$

The above equation to calculate the distance between two CDR3β of two different TCRs can be applied to take into account all CDRs. In this case, all 6 CDRs were described by sliding windows of 4 residues, the distances between each pair of CDR1α, CDR2α, CDR3α, CDR1β, CDR2β, and CDR3β were calculated and summed up to give the distance between the two TCRs.

Of note, when multiple TCRs are to be compared, all distances between each possible pair of TCRs were calculated as described above. These distances were then normalized by dividing each of them by the maximum calculated value for all possible pairs. Consequently, the final distance between two TCRs ranges from 0 (for two totally unrelated TCRs) to 1 (for two TCRs bearing an identical set of 4 residues on each considered loop-only CDR3β or all CDRs).

### Hierarchical clustering of TCRs

Once a normalized distance between each possible pair of TCRs has been calculated, it is possible to use them with the UPGMA (unweighted pair group method with arithmetic mean) algorithm to obtain a hierarchical clustering of all the TCRs.

### Results

(a) Using only CDR3β Loops

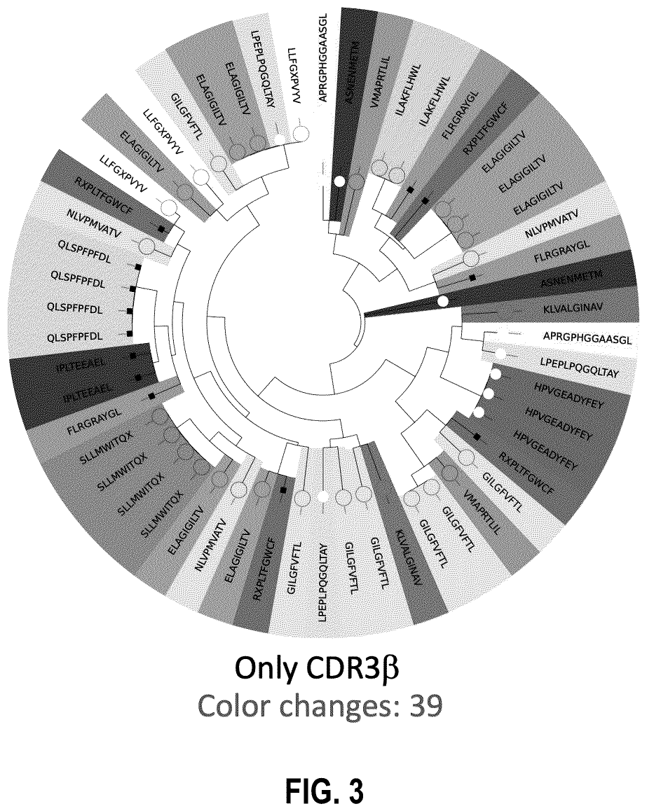

It is generally recognized that the CDR3β loop of a TCR makes most of the interactions with the cognate pMHC. Accordingly, many TCR specificity predictors focus only on the CDR3β sequence. The analysis was first carried out by making the same assumption and clustering the TCRs using an Atchley-based distance calculated on sliding windows of 4 consecutive residues of the CDR3β only of each TCR. The approach was tested using 54 TCRs taken from the Protein Databank, which recognizes 16 different known pMHC. The obtained clustering is depicted in FIG. 3. Note that the clustering shown in FIG. 3 was achieved according to the Atchley-based distance between TCRs CDR3β only. The coloring according to the pMHC bound by the TCR is applied afterwards, only to verify if close TCRs bind identical pMHC, according to the initial objective.

These initial results showed that, even though only CDR3β residues were used to calculate distances, the TCRs have a tendency to spontaneously group to form clusters of TCRs that bind the same pMHC.

As a measure of the quality of the clustering, the number of times the color changes were counted between two consecutive radii, starting from the upper radius of the hierarchical clustering and turning clockwise. 39 color changes were observed, which is a satisfying result in view of the fact that the optimal number of color changes-corresponding to a perfect clustering grouping together all TCRs that bind the same pMHC-is 15, while a random clustering provides an average of 46.9 color changes (p<0.003).

(b) Considering Contribution of all CDRs (α and β Chains)

Based on our previous research in TCR 3D molecular modeling, it was found that the approximation which claims that CDR3β is the most important contributor to TCR specificity is overestimated. As a result, the approach was improved by considering the contribution from all six loops, i.e., CDR1α, CDR2α, CDR3α, CDR1β, CDR2β, and CDR3β.

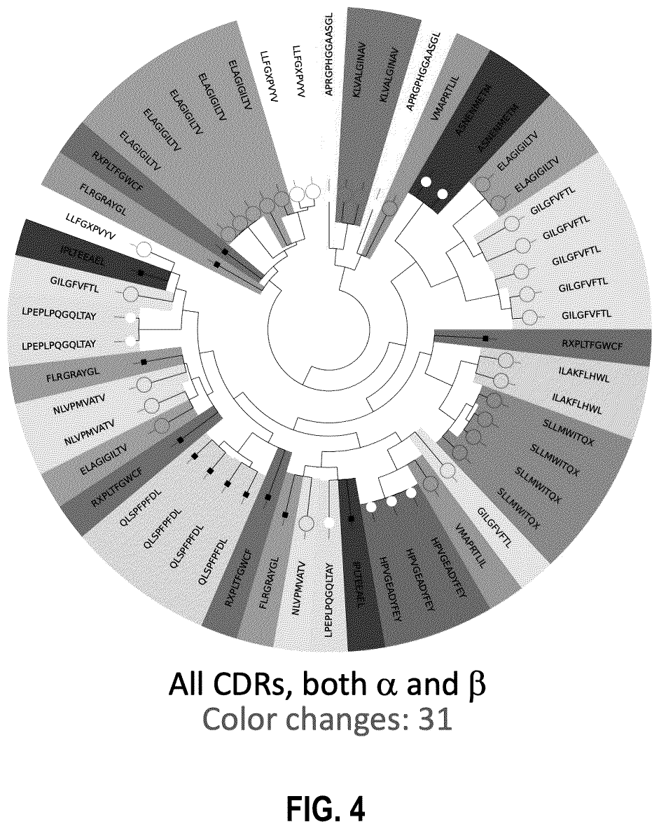

FIG. 4 shows the corresponding hierarchical clustering. Of note, this clustering is much better at grouping together TCRs that bind the same pMHC, with only 31 color changes (p<10⁻⁶ compared to random), compared to 39 when considering only CDR3β (FIG. 3).

(c) Considering Contribution of all CDRs (α and β), as Well as Residue Buriedness

Another route of enhancement that was explored for this approach is to take the buriedness of the CDRs residues into account. Indeed, even though they belong to CDRs, residues that are buried into the TCR structure are not available for interaction with pMHC. Thus, it is possible to enhance the approach by removing the buried residues from those taken into account during the distance calculation.

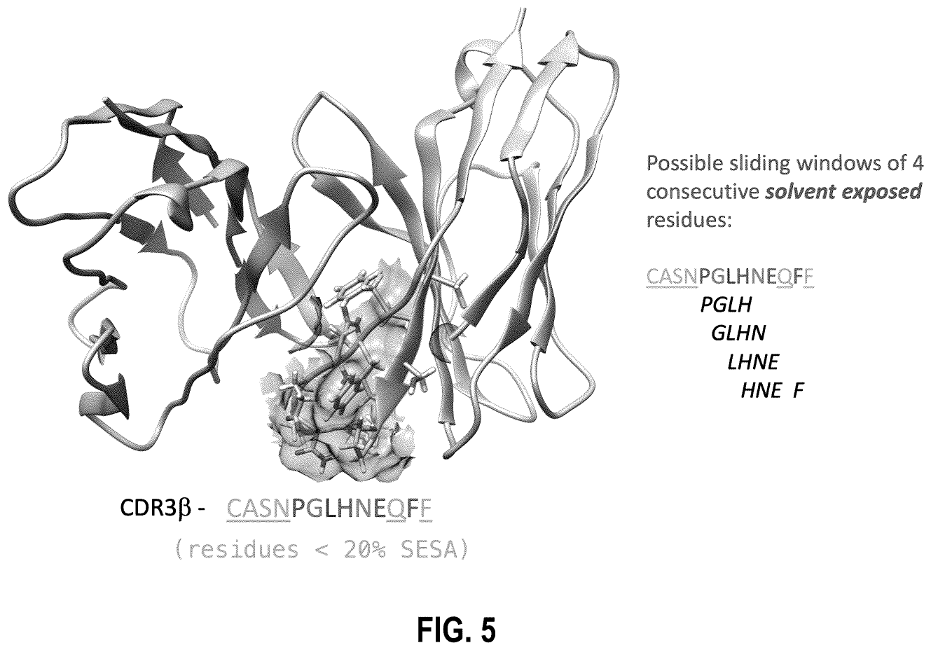

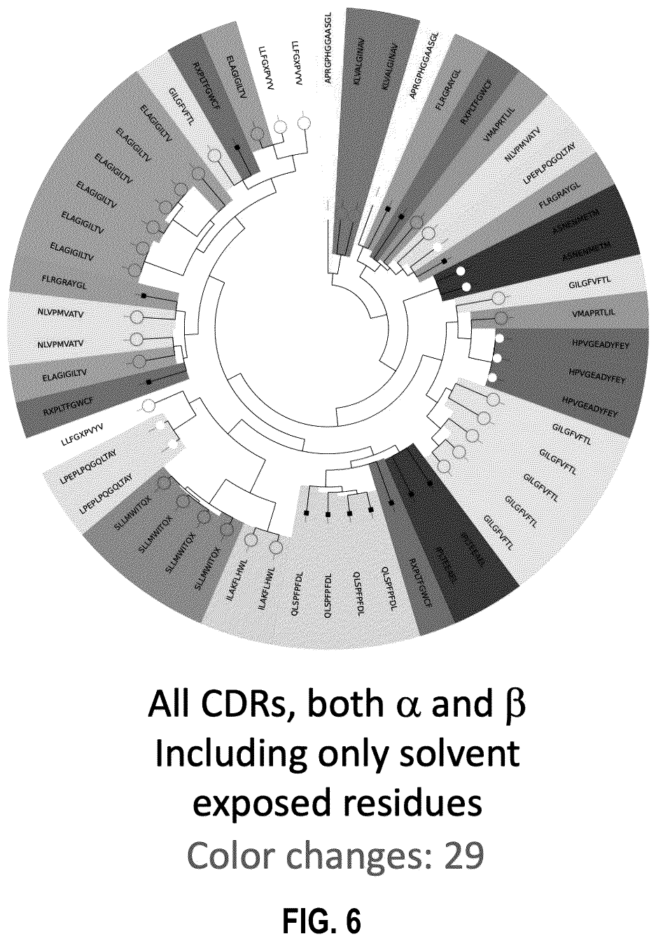

Therefore, the experimental 3D structures of the above-mentioned 54 TCRs were systematically retrieved from the PDB and calculated the Solvent Excluded Surface Area (SESA) for all residues in the 6 CDRs. Then, the residues of CDRs 1 and 2 (α and β), with a SESA>5% of the free, fully exposed amino acid, were considered for distance calculation, while a threshold of 20% was applied for the two CDR3s (α and β). Accordingly, the distances between CDRs were calculated using sliding windows of 4 consecutive solvent-exposed residues and not just consecutive residues (FIG. 5) applied to the first test set of 54 TCRs, taking the buriedness of the residues into account marginally enhanced the quality of the clustering (FIG. 6).

(d) Application to Larger Test Sets

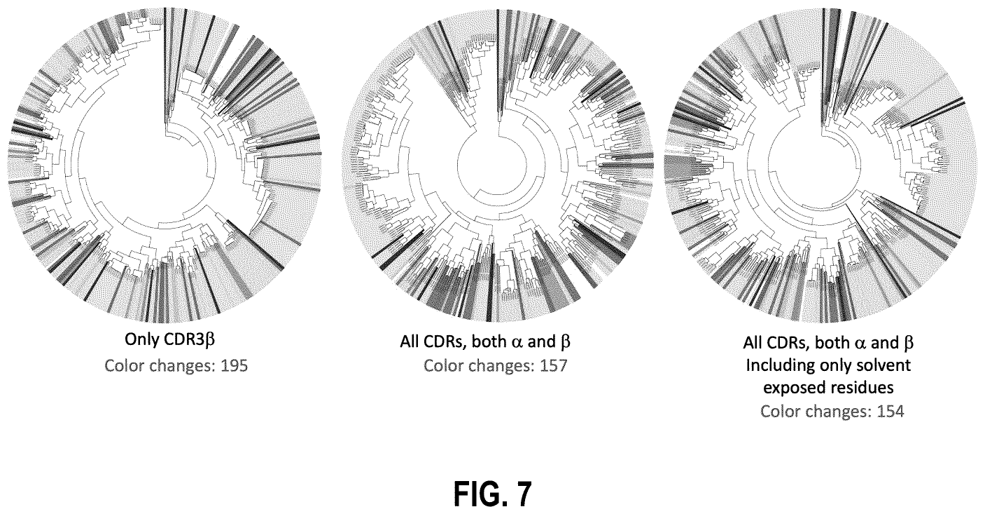

The approach was then applied to a much larger dataset of 374 TCR with known cognate pMHC, taken from VdJdb. Of note, since the experimental 3D structure of these TCRs is unknown, they were modeled by homology modeling to calculate the SESA of the CDRs residues. The results were essentially similar to those obtained from the PDB (FIG. 7), confirming the robustness of the approach.

(e) Discussion

The results obtained on the PDB-extracted dataset and the VDJdb dataset showed that the disclosed clustering approach using an Achtley factor-based distance calculation between the CDRs and TCRs is able to spontaneously group together TCRs that recognize the same pMHC. This approach could therefore be of utmost importance to study TCR repertoires, notably the diversity of specificities from patient samples.

The disclosed approach can be modified to replace the Achtley factors with other physics-based metrics, e.g., BLOSUM matrix, and the weights assigned to the CDR1, CDR2, and CDR3 in the distance calculation can be further optimized. Currently, a weight of 10% is given to each CDR1 and 2 loop, and a weight of 30% is given to each CDR3 loop. Also, the thresholds of buriedness applied to SESA-selected residues can also be optimized. In addition, the number of residues in the above-defined sliding windows can be changed to include a different number of residues, such as 3, 5, 6, etc. (solvent-exposed) residues (e.g., consecutive residues).

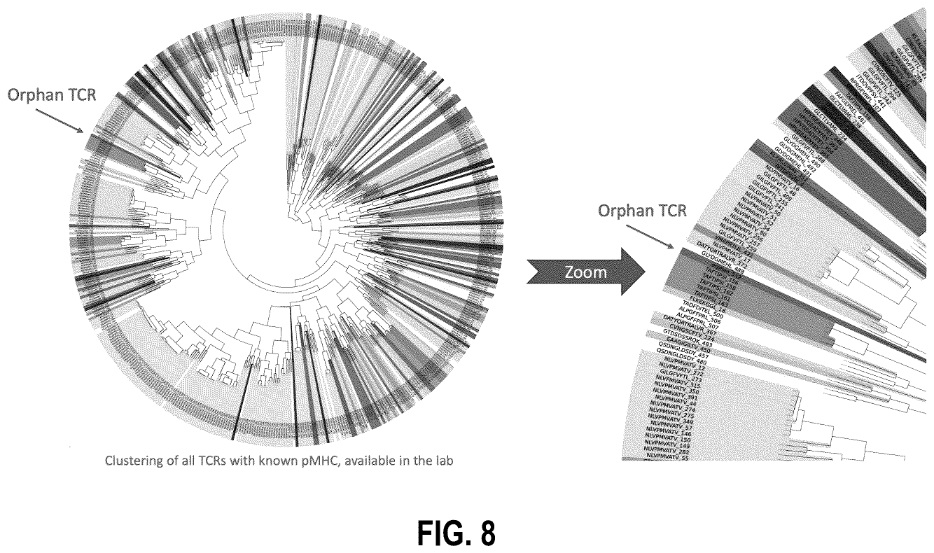

Very importantly, the novel TCR distance calculation and the clustering described herein can be used for the prediction of TCR specificity, i.e., predicting which pMHC a TCR could bind. One possible approach is to cluster a maximum number of TCRs for which the cognate pMHC is known, together with orphan TCRs for which the target is unknown. The positioning of these orphan TCRs indeed provides an indication about possible cognate pMHC, i.e., those bound by the closest TCRs in the hierarchical clustering (FIG. 8).

### Example 3

### Developing and Optimizing pMHC and TCR Structural Fingerprint

Structural fingerprints of the TCR and pMHC are developed following the principle of Electroshape5D. Although the preliminary results obtained using an out-of-the-box version of Electroshape5D and a first biology-driven definition of the centroids provided excellent results, there are several routes of enhancement.

First, other centroids than the Cα of the residues at the center of the 6 CDRs, as defined by the sequence of the latter, are considered. Several biology-driven new centroids are devised, including the center of gravity of the entire TCR surface (i.e., all CDRs), the Cα of the central residue of the HV4 loops, etc. Other centroids than biology-related ones are used. In its small-molecule version, for instance, some centroids of Electroshape5D are defined by cross products of the first ones. In case the first centroids would be accidentally nearly coplanar, such cross products would have the advantage of positioning the next centroids out of the plane, which is necessary to provide a 3D description of the interaction interface. However, one possible drawback of such non-biology-driven centroids is that the meaning of the corresponding entries in the fingerprint vectors of pMHC and TCR could be less correlated. Several definitions are tested to find the best compromise in terms of TCR encoding, clustering, and specificity prediction.

Second, the number of centroids used in the calculation of the fingerprint vectors is increased. Indeed, protein surfaces are substantially larger than the small drug-like molecules for which this approach was initially designed. As a result, encoding a finer description of the protein molecular surface within Electroshape5D may require additional centroids, thus creating additional coordinates in the fingerprint vectors.

Third, an additional dimension is added to the coordinates of the atoms and centroids to provide a more precise definition of the interaction capabilities of the system. Indeed, although the partial atomic charges and atomic contributions to the lipophilicity correlate with the ability of the molecule to exchange electrostatic and non-polar interactions, which are essential for molecular recognition, the current five dimensions lack any explicit information regarding aromaticity. It is recognized, however, that π-π interactions (π-stacking, T-shaped or parallel-displaced π interactions) constitute an important driver of molecular recognition. TCR-pMHC interactions are not an exception to this rule. Therefore, the possibility to add the aromatic character as a 6^{th} dimension to the atom and centroid coordinates is investigated. This aromatic character takes a value of 1 for aromatic atoms and 0 otherwise. Some centroids also have the 6^{th} coordinate set to 1, so the TCR aromatic patches are mathematically translated into the structural fingerprints. Since defining a point in a n-dimension space requires n+1 centroids, adding a dimension to the definition of Electroshape requires introducing at least one more centroid.

Although X-ray structures provide a valuable data set to develop the TCR fingerprint, since they provide both the identity of the pMHC binding partner and the bioactive conformation of the TCR, they may not reflect the real case application where the bioactive conformation of the TCR is unknown. In these real cases, it is necessary to apply the disclosed approach to structural models of TCRs. Whereas the constant part and most of the variable part of the TCRs are structurally conserved across TCRs and can be modeled reliably, the CDRs are particularly flexible, and their preferred (or bioactive) conformation is difficult to predict. Therefore, it is necessary to verify that the fingerprint-based clustering can be applied to structural models with limited loss of predictive ability, i.e., that they are not too sensitive to the exact coordinates used.

Therefore, the following steps are implemented: (i) developing and assessing the fingerprint-based similarity score on the TCR bioactive conformation extracted from experimental TCR-pMHC 3D-structures, (ii) applying it to the same TCRs after modifying their conformation through molecular dynamics (MD) simulations using CHARMM and/or loop conformational sampling algorithms from Rosetta and Modeller, (iii) applying it on homology models of these isolated TCRs to determine the robustness of the approach when confronted to less precise 3D structures, and (iv) applying it comprehensively to TCR homology models obtained for interacting pairs extracted from the VdJdb database.

The goal of steps (ii) and (iii) is to explore the conformational space of the TCR proteins, notably their CDRs. Three sampling engines are used: the loop refinement protocols of Rosetta and Modeller, and MD simulations performed with CHARMM. For the latter, a simulating annealing approach which was found to perform satisfactorily in this context, is used, but more recent and advanced enhanced sampling methods can also be utilized. For example, an implicit continuum model of the solvent during these MD simulations, such as FACTS, can be used instead of explicit water molecules since this would save a considerable amount of computing time—which is necessary for large scale applications of the approach. Once alternative conformations are generated, a limited number of meaningful ones are selected for fingerprint calculations. Several sizes for these collections of conformations: 1, 10, 50, 100, 200, and 500 conformations are tested. The latter is selected based on several possible scoring functions: the Molecular Mechanics-Generalized Born Surface Area (MM-GBSA), which was originally developed for this purpose, the Rosetta scoring function, the Discrete Optimized Protein Energy (DOPE) scoring of Modeller, or the QMEAN scoring of SwissModel. Other combinations of sampling engines and scorings could be devised in response to the need of the project. The combination “sampling engine/scoring function to select the top-ranked conformations used to calculate the fingerprints” is determined so as to optimize the relevance of the similarity measure used in the clustering protocol (see below).

Step (iv) is used to validate the efficiency of the fingerprint-based clustering approach on an external test set after removing from VdJdb all TCR-pMHC complexes whose structures have been experimentally determined.

To assess the relevance of the similarity measure between TCRs, the “sequence recapitulation” is used as a readout. For each TCR of the training set, considered in turn as a reference TCR (TCRref), the closest TCR is calculated from the rest of the training set (excluding TCRref itself). The sequence identity between the peptide epitopes that are known to be recognized by these TCRs is then calculated. For instance, if TCRref was crystallized in complex with prefMHC and the closest TCRclose was crystallized in complex with pcloseMHC, the sequence recapitulation for this pair is the sequence identity between the peptides pref and pclose. This procedure is repeated for each TCR considered in turn as TCRref. The final readout, called sequence recapitulation, for a given fingerprint-based similarity score (resulting from a combination of all of the previous routes of optimization, i.e., number and nature of the centroids, structural origin-X-ray or model-of the CDRs, etc.) is defined as the averaged sequence identity over each TCRref/TCRclose pair. Of note, the HLA-A*02 restricted training set, for example, contains several pMHCs that are recognized by different TCRs, allowing a relevant application of this procedure: the averaged sequence recapitulation of “random” similarity measure is estimated around 32%, while the maximum sequence recapitulation that is possible to obtain is 92%. A sequence recapitulation of 74% was obtained for the experimental structures of TCRs able to bind HLA-A2 restricted epitopes.

All the above-mentioned routes of enhancement of the fingerprint-based similarity can be considered as parameters in an optimization process. As a result, great attention needs to be paid to prevent any overfitting of the final combination. To this end, the robustness of the models is checked through cross validation. This procedure consists in dividing the entire dataset into a training set used to develop the best fingerprint-based similarity score as possible and a test set used to validate its predictive ability. The procedure is repeated several times (typically 100 to 1000) to obtain a statistically relevant estimation of the robustness of the similarity score. This procedure is applied in several projects. In addition, the VdJdb database is used as an independent test set, as mentioned above, to verify that the methodology is not overfitted regarding the training set.

The VdJ database reports several pMHC recognized by tens to hundreds of different TCRs. These cases allow assessment of the ability of the fingerprint-based approach to cluster together TCRs recognizing the same epitope despite potentially large sequence differences. It is hypothesized that large series of different TCRs recognizing the same pMHC could be divided into several clusters, each one potentially corresponding to a different orientation of the TCR with respect to the pMHC. If such multiple TCR clusters per pMHC are found, homology models of the corresponding TCR-pMHC are generated to verify the hypothesis or explore alternative explanations. The number of TCR-pMHC complexes to investigate in these cases is reasonably small (some tens to hundreds) to ensure the feasibility of obtaining these models.

The ability of the structure-based fingerprint methodology to cluster TCRs with similar pMHC specificity is compared with those of the sequence-based approaches (Dash, P., et al. Nature 547, 89-93 (2017); Glanville, J., et al. Nature 547, 94-98 (2017); Ostmeyer, J., et al. Cancer Res. (2019)). The efficiency of the different approaches is analyzed in view of the number of available TCR sequences targeting a given pMHC: the disclosed methodology provides a more meaningful clustering than sequence-based methods when little TCR sequences are available for the training (usual cases in clinics), while sequence-based methods are expected to be faster for cases where a large number of TCRs are available (rare situations of highly-studied ‘archetypal’ epitopes).

The procedure described above for the design of structural fingerprints of TCRs is applied to pMHC. The definition of the pMHC-related centroids is adapted to the particular geometry and interaction profile of these molecules. As for the TCR, pMHC-related fingerprints are designed and tested in successive phases of growing complexity and possible difficulty: pMHC conformation extracted from X-ray structures, from conformational alternative or homology models of the same complexes, and finally from pMHC extracted from VdJdb. Readout and statistical validation of the robustness and predictive ability of the fingerprint-based similarity will be comparable to those mentioned above for TCR.

### Example 4

### Development of Machine Learning Approaches to Predict TCR Specificities

Several types of fingerprints and machine-learning approaches are developed and used for TCR-pMHC matching prediction. First and importantly, entries in the Electroshape vectors are grouped by triplets, each one corresponding to distance distributions of the CDR (or pMHC) atoms to a given centroid at orders 1, 2, and 3. A direct result of this definition is that triplets of fingerprint entries are likely to reflect the characteristics of complementary regions of the TCR and pMHC surfaces in 5D or 6D. For instance, the entries of the TCR fingerprint vector related to the centroids defined as being the center of the CDR1 or CDR2 provide an information complementary (and therefore numerically correlated) to the entries of the pMHC fingerprint related to the centroid defined as being the backbone nitrogen atom of the peptide N-terminus. Reversely, the entries of the TCR fingerprint vector related to the centroids defined as being the center of the CDR1 or CDR2 provide an information complementary (thus correlated) to the entries of the pMHC fingerprint related to the centroid defined as being the backbone carbon atom of the peptide C-terminus carboxylate. In addition, the entries of the TCR fingerprint vector related to the centroids defined as being the center of the CDR3 or CDR3 are correlated to those of the pMHC fingerprint related to the centroid defined as the peptide's center of gravity. Such correlations enable the TCR/pMHC data matching procedure.

Second, the 5D or 6D coordinates used to describe the TCR and pMHC surface are physics-based, providing another source of correlation between the TCR and pMHC structural fingerprints. Indeed, the Cartesian coordinates describe the shape of the TCR and pMHC surfaces, which are complementary when particular TCR and pMHC are real binding partners. The 4th dimension, i.e., the atomic partial charge, correlates between matching TCR and pMHC since charges of opposite signs attract each other while charges of the same sign repulse each other. The 5th dimension, i.e., the atomic contribution to the lipophilicity, reflects that non-polar patches are complementary between matching TCRs and pMHCs. Finally, the possible 6th dimension, i.e., the atomic aromaticity, correlates between matching TCR and pMHC since re-interactions provide additional driving force to the binding strength and specificity.

In the current implementation of their structural fingerprints, some centroids of pMHCs are dedicated to exclusively reflecting the charge distribution of the pMHC surface around them. Such centroids are implemented in the definition of the TCRs structural fingerprints. This helps the data matching process by isolating anti-correlated features (charge) from correlated ones (non-polarity and aromaticity).

Several supervised data matching approaches are adapted, trained, and tested for their ability to detect real TCR and pMHC binding partners from their fingerprints. When inputting a given TCR fingerprint and a given pMHC fingerprint, such approaches should typically provide a desired output (matching decision) value close to 1 for real matching TCR: pMHC pairs and close to 0 otherwise. These approaches are developed using the same training set and test sets used to design the fingerprints as described above.

The conceptual advantages of the designed structural fingerprints, described in the previous paragraphs, encourage the use of simple linear regressions or classifications between the related entries of the TCR and pMHC fingerprints, or of a combination of rule-based approaches that derive the match decision by a logical combination of match conditions. These approaches are tested first, since they provide an intuitive interpretation of the matchings in terms of complementary interactions between the CDR3 α/β and the peptide epitope, or the CDR1 α/β and CDR2 α/β of the TCR and the N or C-termini of the peptide (and of the surrounding MHC). Logistic regressions, naïve Bayes models, perceptrons or support vector machines, using, for instance, the distances between the TCR and pMHC fingerprint entries (term by term) as input, are investigated. Multiple matching algorithms are used to provide the final matching decision in a consensus approach. Several APIs are available to perform these machine-learning investigations, including TensorFlow and Scikit-learn.

As a proof of concept, a correlation coefficient of 0.64 was found between the sequence identity of the peptide presented by a given pMHC and the one recognized by a given TCR on the one hand, and some of the entries of the TCR and pMHC fingerprint vectors on the other hand. Although a correlation coefficient of 0.64 can be seen as insufficient for the final objective, it should be reminded that it has been obtained without any optimization of the pMHC and TCR fingerprints nor of the matching algorithm. This value, therefore, already indicates the effectiveness of the disclosed approaches. This simple multilinear regression shows that the TCR and pMHC fingerprints encode for complementary information can ultimately be used to develop better TCR/pMHC matching algorithms.

Final models are analyzed in terms of specificity and sensitivity. The final models are selected in priority models with high specificity since it is essential to predict real positives for clinical applications. In other words, it is more important to predict pairs of TCR and pMHC that are actually binding experimentally, even at the cost of missing some TCR/pMHC partners, than to try to predict a maximum number of interacting pairs but take the risk to predict also many false positives (i.e., TCR/pMHC predicted to bind, but experimentally found unrelated). Again, cross validation and external test sets are used to ensure the statistical relevance of the robustness and predictive ability of the final approach.

Of note, this structure-based physics-based approach, which capitalizes on known features responsible for molecular recognition, only uses a limited number of parameters that make its training feasible despite the limited amount of data available regarding matching TCRs and pMHCs. This constitutes a significant advantage over other machine learning or deep learning approaches, potentially using only sequence information, which would require very large and currently unavailable training datasets, making them intractable.

### Example 5

### Experimental Validation of the Predicted Results

In order to validate the accuracy of the direct identification of TCR: pMHC pairs from cancer patient samples performed above, three stringent “truth-telling” experiments are performed.

In the first experiment, CD8 T-cell clones of known pMHC specificities for which TCRs were also sequenced (currently available in the library of neoantigen-, tumor-associated antigen- and viral antigen-specific T-cell clones from Harari's group) are used. Three distinct T-cell clones in bulk TILs are spiked at different ratios (e.g., 1:10, 1:100; 1:1000) and run the machine-learning algorithm to challenge the specificity and sensitivity of detection of cognate pMHC (among the top 50 pMHC selected for the patients). This experiment is performed on three independent patients.

The second experiment focuses on known tumor-reactive TCRs obtained from TCR sequencing of CD137-expressing TIL exposed to autologous tumors. A collection of such tumor-reactive orphan TCRs are already available for four patients, and their antitumoral specificity was already validated upon transduction of recipient cells with cloned TCRs. Using an integrated proteo-genomic approach, the top 100 predicted private pMHC are obtained, and direct prediction of TCR: pMHC pairs is applied as output from the machine-learning algorithm developed above to predict the pMHC recognized by the TCRs of the four patients. Multimeric pMHC complexes (or functional assays with synthetic peptides) are then used to validate the predicted TCR-pMHC pairs.

In the third experiment, the fingerprints/machine-learning method developed above is applied to three additional patients in real-world conditions. To this end, m single-cell TCR sequencing (scTCR-Seq, as routinely performed in Harari's lab using the 10Xgenomics platform) is performed on 5,000 bulk TILs and, in parallel, the top 50 potential tumor pMHCs from each patient (as for the aforementioned second experiments) is determined. Then, upon a direct prediction of TCR: pMHC pairs as output from the machine-learning algorithm, a couple of identified TCR: pMHC pairs are selected, and TCR sequences are cloned to transduce autologous bulk primary peripheral blood mononuclear cells. In order to demonstrate the accuracy of the approach, fluorescent pMHC multimers are synthesized to validate TCR specificities by FACS. This experiment unambiguously validates the successful direct identification of TCR:pMHC pairs from cancer patient samples.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A method of identifying two immunological entities as having similar specificity to an epitope, comprising:
(a) selecting a subset of amino acids in a first immunological entity and a corresponding subset of amino acids in a second immunological entity, wherein the subset of amino acids in the first immunological entity and the corresponding subset of amino acids in the second immunological entity have an identical number of amino acids;
(b) determining an amino acid sum of differences in each of a plurality of physicochemical properties by performing a pairwise comparison between an amino acid in the subset of amino acids in the first immunological entity and a corresponding amino acid in the corresponding subset of amino acids in the second immunological entity;
(c) repeating steps (a) to (b) for remaining amino acids in the subset of amino acids in the first immunological entity and the corresponding subset of amino acids in the second immunological entity;
(d) determining a subset sum of differences between the subset of amino acids in the first immunological entity and the corresponding subset of amino acids in the second immunological entity;
(e) repeating steps (a) to (d) for one or more subsets of amino acids in the first immunological entity and the second immunological entity;
(f) determining an aggregate value of all subset sums of differences between the first immunological entity and the second immunological entity by assigning a weight value to each of the subset sums; and
(g) identifying the first immunological entity and the second immunological entity as having similar specificity to an epitope if the aggregate value is smaller than a threshold value.

2. A method of identifying a subset of immunological entities as having similar specificity to an epitope, comprising:
(i) providing a plurality of immunological entities;
(ii) selecting two immunological entities from the set of immunological entities for pairwise comparison;
(iii) identifying the two immunological entities as having similar specificity to an epitope according to the method of claim 1; and
(iv) repeating steps (ii) to (iii) for remaining immunological entities in the plurality of immunological entities and identifying a subset of immunological entities from the plurality of immunological entities as having similar specificity to the epitope.

3. A method of identifying two immunological entities as having similar specificity to an epitope, comprising:
(i) providing a plurality of immunological entities;
(ii) selecting two immunological entities from the set of immunological entities for pairwise comparison;
(iii) performing steps (a) to (f) of the method of claim 1; and
(iv) repeating steps (ii) to (iii) for remaining immunological entities in the plurality of immunological entities and identifying a pair of immunological entities that have a minimum aggregate value among the plurality of immunological entities as the two immunological entities having similar specificity to the epitope.

4. A method of identifying a subset of immunological entities as having similar specificity to an epitope, comprising:
(1) providing a plurality of immunological entities;
(2) generating a similarity matrix for a subset of amino acids of an immunological entity of the set of immunological entities, wherein the similarity matrix comprises a plurality of physicochemical properties of each amino acid in the subset of amino acids;
(3) repeating step (2) for one or more subsets of amino acids of the immunological entity;
(4) repeating steps (2) to (3) for remaining immunological entities of the plurality of immunological entities; and
(5) performing a clustering analysis based on a distance between two corresponding similarity matrices of a pair of immunological entities to identify a subset of immunological entities having similar specificity to the epitope.

5. The method of claim 4, wherein the distance is a Manhattan distance.

6. The method of any one of claims 4-5, wherein the clustering analysis comprises a hierarchical clustering.

7. The method of any one of claims 6, wherein the hierarchical clustering comprises an unweighted pair group method with arithmetic mean (UPGMA).

8. The method of any one of the preceding claims, wherein the immunological entity is a T cell receptor (TCR), a B cell receptor (BCR), an antibody, or a chimeric antigen receptor (CAR).

9. The method of claim 8, wherein the immunological entity is a TCR.

10. The method of claim 8 or 9, wherein the epitope is located on peptide-MHC (pMHC).

11. The method of any one of the preceding claims, wherein the subset of amino acids comprises 3 to 8 amino acids.

12. The method of claim 11, wherein the subset of amino acids comprises 3 to 8 consecutive amino acids.

13. The method of any one of claims 11-12, wherein the subset of amino acids consists of 4 amino acids.

14. The method of any one of claims 9-13, wherein the one or more subsets of amino acids are selected from amino acids in CDR1α, CDR2α, CDR3α, CDR1β, CDR2β, and CDR3β.

15. The method of claim 14, wherein step (f) determining an aggregate value comprises assigning a weight value of about 30% to the subset of amino acids in CDR3α or CDR3β.

16. The method of claim 14, wherein step (f) determining an aggregate value comprises assigning a weight value of about 10% to the subset of amino acids in CDR1α, CDR2α, CDR1β, or CDR2β.

17. The method of any one of the preceding claims, wherein the subset of amino acids does not include amino acids that are not solvent-exposed.

18. The method of claim 17, wherein the subset of amino acids does not include amino acids in CDR1α, CDR2α, CDR1β, or CDR2β that have a relative solvent excluded surface area (SESA) of less than about 5%.

19. The method of claim 17, wherein the subset of amino acids does not include amino acids in CDR3α or CDR3β that have a SESA of less than about 20%.

20. The method of any one of the preceding claims, wherein the physicochemical properties comprise amino acid attributes selected from hydrophilicity value, polar requirement, long range nonbonded energy per atom, negative charge, positive charge, size, normalized relative frequency of bend, normalized frequency of β-turn, molecular weight, relative mutability, normalized frequency of coil, average volume of buried residue, conformational parameter of β-turn, residue volume, isoelectric point, optimized propensity to form reverse turn, chou-fasman parameter of coil conformation, information measure for loop, free energy in β-strand region, side chain volume, amino acid composition of total proteins, average relative probability of helix, α-helix indices, relative frequency of occurrence, helix-coil equilibrium constant, amino acid composition, number of codon(s), net charge, normalized frequency of turn, relative frequency in α-helix, average nonbonded energy per residue, bulkiness, normalized relative frequency of coil, refractivity, normalized frequency of left-handed α-helix, heat capacity, free energy in α-helical region, hydrophobicity factor, normalized frequency of extended structure, normalized frequency of β-sheet, unweighted, normalized frequency of β-sheet, information measure for pleated-sheet, hydropathy index, eisenberg hydrophobic index, average side chain orientation angle, average interactions per side chain atom, transfer free energy, and percentage of buried residues.

21. The method of claim 20, wherein the physicochemical properties comprise hydrophobicity, secondary structure propensity, size/mass, amino acid composition, codon degeneracy, and electrostatic charge.

22. A method of identifying an epitope for a TCR that binds specifically to the epitope, comprising:
identifying a second TCR that has similar specificity to an epitope with the TCR according to the method of any one of claims 1-21, wherein the second TCR binds specifically to a known epitope; and
identifying the known epitope as an epitope to which the TCR binds specifically.

23. A method of identifying one or more TCRs that bind specifically to an epitope, comprising:
selecting a candidate TCR that binds specifically to the epitope;
identifying at least one TCR that has similar specificity to an epitope with the candidate TCR according to the method of any one of claims 1-21; and
identifying the at least one TCR as the one or more TCRs that bind specifically to the epitope.
